(19) United States
(12) Patent Application Publication
(10) Pub. No.: US 2026/0266794 A1
(43) Pub. Date: Sep. 10, 2026

(54) METHOD FOR ANALYZING DEGRADATION OF MARINE BIODEGRADABLE PLASTIC

(71) Applicant: SHIMADZU CORPORATION, Kyoto-shi, Kyoto (JP)

(72) Inventors: Chihiro NOUDA, Kyoto-shi, Kyoto (JP); Kana KUROISHI, Kyoto-shi, Kyoto (JP); Makoto YASOJIMA, Kyoto-shi, Kyoto (JP); Hiroaki TAKEMORI, Kyoto-shi, Kyoto (JP)

(73) Assignee: SHIMADZU CORPORATION, Kyoto-shi, Kyoto (JP)

(21) Appl. No.: 19/162,918

(22) PCT Filed: Apr. 26, 2024
(86) PCT No.: PCT/JP2024/016375

§ 371 Date: Sep. 5, 2025

(30) Foreign Application Priority Data
Mar. 8, 2023 (JP) 2023-035759

(51) Int. Cl. G01N 33/18; G01N 27/62

(52) U.S. Cl. CPC G01N 33/18; G01N 27/62

(57) ABSTRACT

The present disclosure provides a method for analyzing the degradation of a marine biodegradable plastic.

### TECHNICAL FIELD

The present invention relates to a method for analyzing the degradation of a marine biodegradable plastic.

### BACKGROUND ART

In response to the problem of marine plastic waste, the demand for marine biodegradable plastics, which are degraded by microorganisms in the ocean, is increasing, and product development is progressing. Specifically, a biodegradable polyester composed of a polymer of 3-hydroxybutyric acid and 3-hydroxyhexanoic acid has been proposed and is commercially available (see Patent Literature 1).

For such marine biodegradable plastics, the elucidation of the biodegradation mechanism, which is necessary information for product development according to the application, has not progressed. In conventional methods, the biodegradation of marine biodegradable plastics is evaluated by methods such as quantifying the amount of gas consumed as decomposition proceeds (ISO 23977-2) and comparing the weight of the plastic before and after biodegradation (ISO 22766).

### PRIOR ART DOCUMENTS

### Patent Literature

[Patent Literature 1] Japanese Unexamined Patent Application Publication No. 2007-SUMMARY OF INVENTION

### Technical Problem

However, since conventional methods are all indirect methods, it is not possible to grasp what kind of degraded intermediates are generated and how they are distributed during the degradation process. Grasping such information on degraded intermediates will help elucidate the degradation mechanism, and therefore the development of a new analysis method is required.

An object of the present invention is to provide an analysis method that can provide information on the degradation mechanism of a marine biodegradable plastic.

### Solution to Problem

An analysis method according to a first aspect of the present invention is a method for analyzing degradation of a marine biodegradable plastic, the method comprising an extraction step of eluting and extracting a degradation intermediate product adhered to the marine biodegradable plastic and/or a biofilm formed on a surface of the marine biodegradable plastic into a solvent, and a detection step of performing mass spectrometry using a solution in which the degradation intermediate product is extracted to detect the degradation intermediate product.

### Advantageous Effects of Invention

According to the analysis method of the first aspect, it is possible to grasp the presence or absence and the degree of polymerization of degraded intermediates generated during the degradation of the marine biodegradable plastic, which can be useful for elucidating the degradation mechanism.

### BRIEF DESCRIPTION OF DRAWINGS

FIG. 1 is a flowchart showing the experiment in Example 1.

### DESCRIPTION OF EMBODIMENTS

### 1. First Embodiment

The analysis method of the first embodiment of the present invention is a method for analyzing the degradation of a marine biodegradable plastic, specifically, its degraded intermediates, and comprises a preparation step, an extraction step, and a detection step in order. Hereinafter, each step will be described in detail.

### Preparation Step

In this step, a marine biodegradable plastic with a biofilm attached to its surface is prepared as an analysis sample.

The marine biodegradable plastic may be any polymer that degrades in seawater, and examples thereof include polyhydroxyalkanoates. Examples of monomers constituting the polyhydroxyalkanoate include 3-hydroxybutyric acid, 3-hydroxyisopentanoic acid, 3-hydroxyhexanoic acid, 3-hydroxyoctanoic acid, 3-hydroxydecanoic acid, 3-hydroxyundecanoic acid, 3-hydroxytetradecanoic acid, and 3-hydroxyhexadecanoic acid.

In the first embodiment, a typical example of the analysis sample of the marine biodegradable plastic is a polymer having a structure represented by the following chemical formula (1), that is, a copolymer of 3-hydroxybutyric acid and 3-hydroxyhexanoic acid. In the formula, x represents an integer of 1 or more, and y represents an integer of 1 or more.

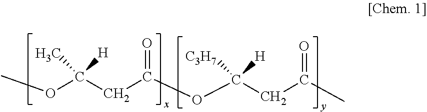

The marine biodegradable plastic with a biofilm formed on its surface (hereinafter referred to as “biofilm-attached plastic”) is obtained by bringing the marine biodegradable plastic into contact with seawater for a certain period of time or longer. Specifically, it may be a marine biodegradable plastic that has been floating or sedimented in the sea for a certain period of time; or a marine biodegradable plastic that has been brought into contact with natural or artificial seawater for a certain period of time (that is, a marine plastic subjected to a biodegradation test). Generally, a marine biodegradable plastic is degraded by microorganisms in seawater, and a biofilm, which is an aggregate of these microorganisms, is formed on its surface. When this biofilm-attached plastic is taken out of the seawater, it has a structure comprising the marine biodegradable plastic, the biofilm formed on its surface, and pore water contained between them.

(Extraction Step) In this step, (1) the marine biodegradable plastic in the process of degradation and (2) the biofilm formed on the surface of at least one of the marine biodegradable plastics are brought into contact with a solvent. As a result, the degraded intermediates attached to the marine biodegradable plastic and/or the biofilm are extracted into the solvent.

Specifically, first, as a pretreatment, a removal treatment such as centrifugation is performed on the biofilm-attached plastic in order to remove excess water such as pore water.

By performing centrifugation, it is separated into a residue and a supernatant. Since a large amount of biofilm and marine biodegradable plastic are contained in the residue, the residue is used. In addition, since a small amount of biofilm remains in the supernatant, in order to enable more accurate quantification, the supernatant may be filtered, and the residue remaining on the surface of the filter paper may be added to the residue in the above centrifugation.

Next, solvent extraction is performed on the residue. That is, the residue including the marine biodegradable plastic and/or the biofilm is brought into contact with the solvent, for example, by immersing the residue in the solvent. At this time, it is preferable to perform ultrasonic extraction in order to surely elute the degraded intermediates attached to the surface of the marine biodegradable plastic and the surface of the biofilm into the solvent.

Examples of the solvent include organic solvents. Among organic solvents, water-soluble solvents such as methanol, ethanol, and 1-propanol are preferable from the viewpoint that they have good affinity with the surface of the biofilm containing water and can elute more degraded intermediates into the solvent.

After the solvent extraction, unnecessary substances in the solvent may be removed by filtration or the like, if necessary.

As a result, a solution in which the degraded intermediates are extracted, that is, a biofilm extract, is obtained.

In addition, when performing liquid chromatography-mass spectrometry in the detection step described later, in order to ensure component separation by liquid chromatography, it is preferable to perform replace to the solvent used in liquid chromatography after solvent extraction in this extraction step. In the first embodiment, this replaced solution (re-dissolved solution) is also included in the “solution in which the degraded intermediates are extracted.”

The replace is performed by evaporating the solution in which the degraded intermediates have been extracted by the above solvent extraction to dryness, and then re-dissolving the dried product in another solvent.

Examples of the other solvent for re-dissolving include solvents used for the mobile phase of liquid chromatography, which are appropriately determined according to the liquid chromatograph apparatus. Specific examples include acidic aqueous solutions such as formic acid aqueous solution, acetic acid aqueous solution, and phosphoric acid aqueous solution; and organic solvents such as methanol, ethanol, acetonitrile, and hexane.

The degradation intermediate product extracted in this step is a compound eluted by the degradation of the marine biodegradable plastic, and is a monomer and/or an oligomer constituting the marine biodegradable plastic, and in particular, both a monomer and an oligomer constituting the marine biodegradable plastic.

For example, when the marine biodegradable plastic is a polymer having a structure represented by chemical formula (1), the monomers are 3-hydroxybutyric acid and 3-hydroxyhexanoic acid. The oligomer is an oligomer composed of at least one of these monomers, and is, for example, represented by the following chemical formula (2). In the formula, x represents, for example, an integer of 1 or more and 15 or less, and y represents, for example, an integer of 1 or more and 15 or less. The total degree of polymerization of the oligomer, that is, the total value of x and y, is, for example, 20 or less, preferably 12 or less, and, for example, 1 or more, although it depends on the type of mass spectrometer, when a liquid chromatograph-tandem mass spectrometer is used as the mass spectrometer.

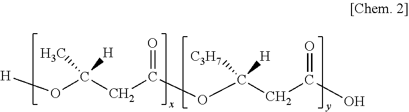

(Detection Step) In this step, mass spectrometry is performed using the solution obtained in the extraction step, that is, the solution in which the degraded intermediates are extracted. As a result, the degraded intermediates of the marine biodegradable plastic are detected.

Preferably, liquid chromatography-mass spectrometry is performed as the mass spectrometry method. That is, after the solution in which the degraded intermediates are extracted is subjected to component separation by liquid chromatography, mass spectrometry is performed.

In liquid chromatography, any known or conventional column can be appropriately selected as the column to be used, as long as it can retain the monomers and oligomers constituting the marine biodegradable plastic. For example, a column packed with octadecyl group-containing silica gel as a stationary phase can be mentioned. The mobile phase can be appropriately selected according to the column and the marine biodegradable plastic, and examples thereof include the above-mentioned solvents, and a mixed solvent of an acidic aqueous solution and an organic solvent is preferable.

In the first embodiment, a gradient elution method is preferably adopted. That is, the concentration of the organic solvent in the mobile phase is gradually increased from the beginning of the analysis. This makes it possible to efficiently separate multiple types of degraded intermediates having a wide molecular weight distribution and shorten the run time.

In mass spectrometry, examples of the ionization means include Electron Ionization (EI), Electrospray Ionization (ESI), and Atmospheric Pressure Chemical Ionization (APCI).

Examples of the type of mass separation, that is, the type of mass spectrometer, include a quadrupole mass spectrometer, a magnetic sector mass spectrometer, a time-of-flight mass spectrometer, an ion trap (including Orbitrap) mass spectrometer, and an ion cyclotron resonance mass spectrometer. A tandem mass spectrometer composed of a plurality of analyzers may also be used. That is, in the first embodiment, a liquid chromatograph-tandem mass spectrometer (LC/MS/MS) may be adopted. Examples of the type of tandem mass spectrometry (MS/MS) include a triple quadrupole (Q-Q) mass spectrometer, a tandem time-of-flight (TOF-TOF) mass spectrometer, a quadrupole-time-of-flight (Q-TOF) mass spectrometer, a quadrupole-ion trap (Q-IT) mass spectrometer, a quadrupole-ion cyclotron resonance (Q-ICR) mass spectrometer, and an ion trap-time-of-flight (IT-TOF) mass spectrometer. From the viewpoint of being able to measure the accurate mass and easily and accurately distinguishing and identifying similar degraded intermediates from each other, it is preferable to use an ion trap mass spectrometer, a time-of-flight mass spectrometer, or an ion cyclotron resonance mass spectrometer. From the viewpoint of easily and accurately quantifying each degradation intermediate product, it is preferable to use a quadrupole mass spectrometer, particularly a triple quadrupole mass spectrometer.

As the liquid chromatograph-mass spectrometer used for liquid chromatography-mass spectrometry, a conventional or commercially available one may be used. Various setting conditions for the liquid chromatograph and mass spectrometry may be appropriately set in accordance with the recommendations of various apparatuses, with reference to the composition of the monomers and oligomers in the degraded intermediates.

As a result, the degraded intermediates are detected. That is, a mass spectrum is obtained for each degradation intermediate product separated by liquid chromatography. In the mass spectrum, the m/z and peak intensity of ions derived from the monomers and/or oligomers constituting the degradation intermediate product (precursor ions, fragment ions, or product ions) are output. Then, in each of these mass spectra, each monomer and each oligomer constituting the degradation intermediate product can be identified based on the m/z and peak intensity of each ion.

In this step, the degree of polymerization of the degraded intermediates, that is, various oligomers, can also be determined. That is, by calculating and associating the m/z of these oligomers in advance based on the structure and molecular weight of the degraded intermediates, that is, various oligomers, and the structure and molecular weight of the monomers constituting the oligomers, detecting the peaks of ions derived from the monomers and/or oligomers constituting the degraded intermediates in the mass spectrum obtained in the detection step, and collating them with the corresponding m/z, the degree of polymerization of each oligomer can be determined.

According to the analysis method of the first embodiment, the degraded intermediates of a marine biodegradable plastic can be analyzed. It has been conventionally thought that when a marine biodegradable plastic is degraded in seawater, its degraded products are immediately assimilated or mineralized, or do not exist at a detectable concentration level due to dilution by seawater. In contrast, the present inventors focused on the biofilm adhering to the marine biodegradable plastic, predicted that degraded intermediates may exist at a measurable concentration level within the biofilm, which is the site where biodegradation is actually progressing, and actually measured the surface of the biofilm with a mass spectrometer, confirming that a large amount of degraded intermediates are adhered, leading to the invention of the first embodiment.

In particular, according to the analysis method of the first embodiment, it is possible to grasp the presence or absence and the degree of polymerization of degraded intermediates generated during the degradation of the marine biodegradable plastic, which can be useful for elucidating the degradation mechanism. Furthermore, by using this analysis method in parallel with conventional biodegradation test, it becomes possible to consider the relationship between the degree of biodegradation and degraded intermediates, which may lead to the proposal of a new biodegradation degree evaluation method.

### 2. Aspects

Those skilled in the art will understand that the exemplary embodiments described above are specific examples of the following aspects.

(Item 1) An analysis method according to one aspect may be a method for analyzing degradation of a marine biodegradable plastic, the method comprising an extraction step of eluting and extracting a degradation intermediate product adhered to the marine biodegradable plastic and/or a biofilm formed on a surface of the marine biodegradable plastic into a solvent, and a detection step of performing mass spectrometry using a solution in which the degradation intermediate product is extracted to detect the degradation intermediate product.

(Item 2) In the analysis method according to item 1, the extraction step may comprise removing pore water present in the biofilm and then bringing the biofilm into contact with the solvent.

(Item 3) In the analysis method according to item 1 or 2, the solvent may be an organic solvent.

(Item 4) In the analysis method according to any one of items 1 to 3, the degradation intermediate product may be a monomer and/or an oligomer constituting the marine biodegradable plastic.

(Item 5) In the analysis method according to item 4, the degradation intermediate product may be a monomer and an oligomer constituting the marine biodegradable plastic.

(Item 6) In the analysis method according to item 4 or 5, the oligomer may be an oligomer having a total degree of polymerization of up to 20.

(Item 7) In the analysis method according to item 6, the oligomer may be an oligomer having a total degree of polymerization of up to 12.

(Item 8) In the analysis method according to any one of items 1 to 7, in the detection step, the solution in which the degradation intermediate product is extracted may be subjected to component separation by liquid chromatography, and then mass spectrometry may be performed.

(Item 9) In the analysis method according to item 8, the liquid chromatography may be performed by gradient elution.

### EXAMPLES

Next, the present invention will be described in detail with reference to Examples and Comparative Examples, but the scope of the present invention is not limited thereto.

### Example 1

To seawater collected in Osaka Bay, ammonium chloride was added to a concentration of 19.1 mg/L and disodium hydrogen phosphate to 2.3 mg/L to prepare test seawater, and 300 mL of the test seawater was poured into each of fifteen 500 mL glass test bottles. Fifteen sheets of Kaneka biodegradable biopolymer Green Planet™ (registered trademark) (100 μm thick, 30 mg) represented by the following chemical formula were prepared as marine biodegradable plastic films, and one film was immersed in each of the above-mentioned test bottles. These were maintained at a water temperature of 27° C. and left to stand while stirring the test seawater for a predetermined period (5 days, 8 days, 9 days, 10 days, 13 days). During this period, the formation of a biofilm on the surface of the plastic film was visually confirmed.

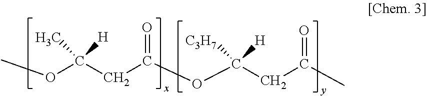

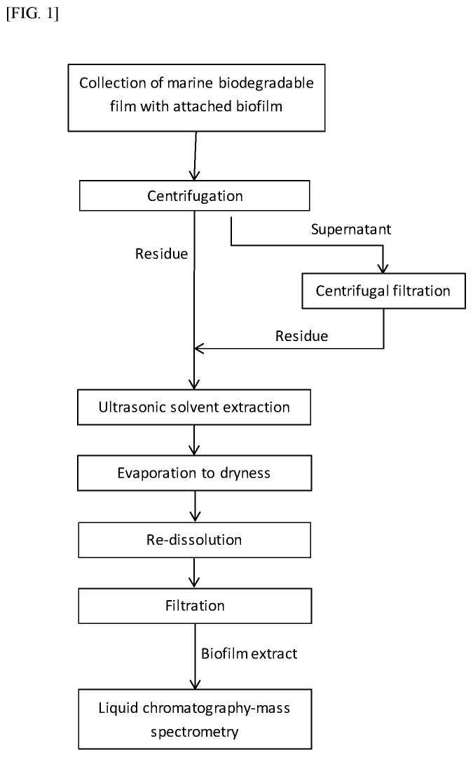

After each predetermined period had elapsed, three plastic films with biofilms formed thereon were taken out and centrifuged at 1000 g for 20 minutes to separate them into a residue and a supernatant. The supernatant was subjected to centrifugal filtration at 2000 g for 20 minutes using a PTFE filter (trade name “Ultrafree-CL”, pore size 0.45 μm), and the residue attached to the filter was collected. These two residues were combined and placed in 8 mL of 1-propanol, and solvent extraction was performed by ultrasonication for 10 minutes. As a result, the components attached to the surface of the marine biodegradable plastic film and the surface of the biofilm were eluted and diffused into the solvent. Subsequently, after removing the marine biodegradable plastic film and the biofilm, the obtained solvent was filtered using a PTFE filter (trade name “GL Chrodisk 13P”, pore size 0.45 μm) to collect the filtrate. The collected filtrate was concentrated to dryness under a nitrogen stream at 30° C. The dried product was dissolved (re-dissolved) in a 0.1% formic acid aqueous solution, and then the re-dissolved solution was filtered using a PTFE filter (trade name “GL Chrodisk 13A”, pore size 0.2 μm) to obtain a biofilm extract as the filtrate (see FIG. 1).

The obtained biofilm extract was analyzed using a liquid chromatograph/Orbitrap mass spectrometer under the measurement conditions shown in Table 1 below. That is, the extract was subjected to component separation by liquid chromatography, and the separated extract was subjected to component analysis by the Orbitrap mass spectrometer.

TABLE 1

| | |
|---|---|
| Measuring Instruments | LC part: Shimadzu Corporation |
| | LC-30AD |
| | MS part: Thermo Fisher |
| | Scientific Q Exactive HF |

LC Part Conditions

| Column | YMC, YMC-Triart C18 | |
|---|---|---|
| | (100 mm × 2.0 mm (id), 3 μm) | |
| Column Temperature | 40° C. | |
| Flow Rate | 0.2 mL/min | |
| Sample Injection Volume | 5 μL | |
| Mobile Phase | A: 0.1% formic acid aqueous solution | |
| | B: 0.1% formic acid/methanol | |
| Gradient Conditions | 0-1 min | A: 90% B: 10% |
| | 1-61 min | A: 90→ 0% B: 10→ 100% |
| | 61-76 min | A: 0% B: 100% |

MS Part Conditions

| | |
|---|---|
| Ionization Method | ESI-Negative |
| Resolution | 60,000 |
| Mass Scan Range | m/z 50-750, 160-2400 |

The obtained qualitative and relative quantitative results are shown in Table 2 below. At this time, for the calculation of the relative quantification of each monomer and each oligomer, the peaks for the standard products of 3-hydroxybutyric acid and 3-hydroxyhexanoic acid (concentration: 200 ng/ml each) were used as a reference. In addition, the presence or absence of a peak in the extracted ion chromatogram was confirmed under the condition of a mass error of +5 ppm for the monoisotopic accurate masses of monovalent and divalent ions calculated from the molecular formulas of the degraded intermediates (oligomers with a total degree of polymerization of up to 20, consisting of two types of monomers: 3-hydroxybutyric acid and 3-hydroxyhexanoic acid). The numbers in Table 2 represent the amount of substance (ng) in the biofilm extract. In the compound column, the number following B indicates the degree of polymerization of 3-hydroxybutyric acid, and the number following H indicates the degree of polymerization of 3-hydroxyhexanoic acid. For example, B1H2 indicates an oligomer in which one 3-hydroxybutyric acid and two 3-hydroxyhexanoic acids are polymerized.

TABLE 2

| Total Degree of | | | Sampling Duration(Day) and Relative Amount | | | | |
|---|---|---|---|---|---|---|---|
| Polymerization | Compound | MW | Day 7 | Day 8 | Day 9 | Day 10 | Day 13 |
| 1 | B1H0 | 104.0 | 608.0 | 1111.5 | 4657.9 | 3278.9 | 3358.5 |
| 2 | B2H0 | 190.1 | 38.6 | 118.2 | 472.9 | 209.4 | 156.2 |
| 3 | B3H0 | 276.1 | 25.7 | 66.7 | 233.8 | 119.6 | 112.4 |
| 4 | B4H0 | 362.2 | 3.7 | 23.4 | 60.6 | 50.6 | 52.8 |
| 5 | B5H0 | 448.2 | 8.1 | 13.6 | 25.0 | 39.4 | 36.5 |
| 6 | B6H0 | 534.2 | 5.2 | 9.0 | 15.3 | 21.5 | 20.8 |
| 7 | B7H0 | 620.3 | 4.5 | 7.3 | 10.5 | 16.6 | 14.0 |
| 8 | B8H0 | 706.3 | 6.0 | 9.9 | 13.3 | 20.3 | 14.3 |
| 9 | B9H0 | 792.3 | 1.8 | 10.5 | 12.2 | 16.5 | 9.0 |
| 10 | B10H0 | 878.4 | | 1.7 | 9.7 | 1.3 | |
| 11 | B11H0 | 964.4 | | | 5.3 | | |
| 12 | B12H0 | 1050.5 | | | 2.1 | | |
| 2 | B1H1 | 218.1 | 13.5 | 27.3 | 68.3 | 38.0 | 20.6 |
| 3 | B1H2 | 332.2 | 5.5 | 7.1 | 22.4 | 12.9 | 6.8 |
| 3 | B2H1 | 304.2 | | | 140.4 | | |
| 4 | B1H3 | 446.3 | 1.6 | 2.6 | 9.1 | 5.1 | 1.4 |
| 4 | B2H2 | 418.2 | 18.3 | 20.4 | 42.8 | 24.6 | 14.5 |
| 4 | B3H1 | 390.2 | 28.6 | 54.7 | 150.0 | 65.5 | 70.0 |
| 5 | B2H3 | 532.3 | | 3.2 | 8.0 | 2.2 | |
| 5 | B3H2 | 504.3 | 19.4 | 28.3 | 37.2 | 24.6 | 17.6 |
| 5 | B4H1 | 476.2 | 21.3 | 42.7 | 73.1 | 43.7 | 60.2 |
| 6 | B3H3 | 618.3 | | | 3.9 | | |
| 6 | B4H2 | 590.3 | 2.8 | 10.4 | 30.3 | 8.0 | 4.8 |
| 6 | B5H1 | 562.3 | 5.5 | 13.3 | 21.8 | 14.1 | 17.3 |
| 7 | B3H4 | 732.4 | | | | 2.2 | |
| 7 | B5H2 | 676.3 | | | 5.0 | | |
| 7 | B6H1 | 648.3 | | 3.8 | 6.1 | 4.7 | 4.6 |
| 8 | B7H1 | 734.3 | | | 3.5 | | |
| 9 | B8H1 | 820.4 | | | 2.3 | | |
| 10 | B9H1 | 906.4 | | | 1.2 | | |
| 1 | BOH1 | 132.1 | 52.1 | 149.4 | 375.6 | 250.5 | 203.7 |
| 2 | BOH2 | 246.1 | 1.0 | 2.2 | 4.3 | 2.5 | 1.3 |
| 3 | BOH3 | 360.2 | | | 0.6 | | |

| | | | | | |
|---|---|---|---|---|---|
| Total Number | 20 | 23 | 32 | 24 | 21 |
| Total Amount [ng] | 870 | 1700 | 6500 | 4300 | 4200 |

### Comparative Example 1

In Example 1, the supernatant seawater in the test bottle into which the marine biodegradable plastic film and the test seawater were injected was collected after a predetermined period (5 days, 8 days, 9 days, 10 days, 13 days), filtered using a PTFE filter, and the filtrate was used as the extract of Comparative Example 1. The extract of Comparative Example 1 was analyzed using a liquid chromatograph/Orbitrap mass spectrometer under the same conditions as in Example 1, but no degraded intermediates of the marine biodegradable plastic could be detected.

As is clear from Table 2, monomer and oligomer components constituting the marine biodegradable plastic were detected in an analyzable amount from the biofilm extract. This proves that degraded intermediates eluted by the degradation of the marine biodegradable plastic film remain on the surface of the biofilm and the surface of the marine biodegradable plastic, and that they can be detected. Therefore, it can be seen that by implementing the analysis method of the first embodiment, information such as the degraded products of the marine biodegradable plastic and their amounts can be obtained.

1. A method for analyzing degradation of a marine biodegradable plastic, the method comprising:
an extraction step of eluting and extracting a degradation intermediate product adhered to the marine biodegradable plastic and/or a biofilm formed on a surface of the marine biodegradable plastic into a solvent; and
a detection step of performing mass spectrometry using a solution in which the degradation intermediate product is extracted, to detect the degradation intermediate product.

2. The analysis method according to claim 1, wherein the extraction step comprises: removing pore water present in the biofilm, and then bringing the biofilm into contact with the solvent.

3. The analysis method according to claim 1, wherein the solvent is an organic solvent.

4. The analysis method according to claim 1, wherein the degradation intermediate product is a monomer and/or an oligomer constituting the marine biodegradable plastic.

5. The analysis method according to claim 4, wherein the degradation intermediate product is a monomer and an oligomer constituting the marine biodegradable plastic.

6. The analysis method according to claim 4, wherein the oligomer is an oligomer having a total degree of polymerization of up to 20.

7. The analysis method according to claim 6, wherein the oligomer is an oligomer having a total degree of polymerization of up to 12.

8. The analysis method according to claim 1, wherein in the detection step, the solution in which the degradation intermediate product is extracted is subjected to component separation by liquid chromatography, and then mass spectrometry is performed.

9. The analysis method according to claim 8, wherein the liquid chromatography is performed by gradient elution.
